(19) United States
(12) Patent Application Publication
(10) Pub. No.: US 2022/0266048 A1
(43) Pub. Date: Aug. 25, 2022

(54) MICROWAVE APPARATUS AND METHOD

(71) Applicant: Emblation Limited, Alloa (GB)

(72) Inventors: Gary Beale, Alloa (GB); Eamon McErlean, Alloa (GB); Matthew Kidd, Alloa (GB); Shailesh Joshi, Alloa (GB)

(73) Assignee: Emblation Limited, Alloa (GB)

(21) Appl. No.: 17/629,030

(22) PCT Filed: Jul. 23, 2020
(86) PCT No.: PCT/EP2020/070814

§ 371 Date: Jan. 21, 2022

(30) Foreign Application Priority Data
Jul. 23, 2019 (GB) 1910527.9

(51) Int. Cl. A61N 5/02; A61B 18/18

(52) U.S. Cl. CPC A61N 5/025; A61B 18/1815; A61B 2018/183; A61B 2018/1861; A61B 2018/0022

(57) ABSTRACT

A microwave system comprises: a microwave generator; a controller configured to control the microwave generator to generate microwave energy having a selected operational frequency or range of frequencies; a microwave cable configured to deliver the microwave energy to a microwave antenna extending from or coupled to a distal end of the microwave cable; and the microwave antenna, the microwave antenna comprising a plurality of slots distributed along a portion of the microwave antenna, wherein a width of the slots varies with distance along the portion of the microwave antenna.

### FIELD

The present invention relates to a microwave apparatus and method, for example a microwave apparatus and method for treating cardiovascular diseases.

### BACKGROUND

In most energy-based treatment systems, such as electromagnetic (EM) ablation systems using microwaves, radiation is delivered from a radiation generator, via a connecting cable, to a radiation delivering applicator placed in or on a tissue.

A method and system for treating cardiovascular diseases (CVD) using such microwave energy system is hereby described.

CVD are broadly divided into two spectrums: coronary and peripheral. Coronary heart diseases occur when the flow of blood to the heart is compromised. Peripheral vascular disease (PVD) arise due to blockages and other complications in the blood vessels of the limbs. CVD can be further divided into two types: Organic and Functional. Organic

CVD is caused by events such as inflammation, plaques, atheroma, blood clots and other tissue damage resulting in the blood vessel structural changes. Functional CVD relates to the functional malfunctioning of the blood vessels without apparent change in their structure. Organic CVD comprises coronary artery disease (CAD), peripheral arterial disease (PAD) and peripheral venous disease (PvD). The most commonly affected coronary arteries are left coronary artery (LCA), right coronary artery (RCA) and left anterior descending artery (LAD). Two typical subtypes of the PAD are a) proximal disease, that comprises aortoiliac and femoropopliteal sites, and b) distal disease, which involves the infrapopliteal sites. Along with the vascular disease, distal PAD may also be accompanied by calcification of the medial layer, that is associated with high mortality. PvD similar to PAD can occur anywhere in the body but mainly affects arms and legs. Venous thromboembolisms (VTE) or blood clots in veins is one of the key factors in causing PvDs and when the clot is deep inside muscles it is called deep vein thrombosis (DVT). Varicose veins are another form of venous complication where damaged valves cause veins in the legs to swell resulting in pain, numbness and weakness in the affected area. CVD when left untreated can result in serious complications such as critical limb ischemia (CLI), pulmonary embolism (PE), stroke and mortality. PVD in the arm has relatively lower incidences as compared to the legs. Blocked axillary artery, brachial artery or radial and ulnar arteries can cause pain, numbness and, similar to the PVD in legs, can lead to increased risk of amputation, heart attack and stroke.

After failed conservative treatments such as lifestyle changes, exercise and pharmacotherapy, revascularisation is typically the only choice left in attempts to treat CVD. Revascularisation involves either widening the blood vessel using balloon angioplasty followed by stent placement also known as PCI (percutaneous coronary intervention) when dealing with coronary arteries; or eventually bypassing the diseased vessel by a graft placement. In general, balloon angioplasty technique involves image guided procedures where a catheter is placed in the diseased artery commonly via either the femoral artery in the groin, or the radial artery in the arm. A guidewire is used through this catheter to position the balloon in the correct place. The balloon filled with contrast agent is inflated to compress the plaque, expand the stent and to reduce the narrowing of the lumen. Compared to the invasive bypass surgery, balloon angioplasty is minimally invasive, offers reduced complications and provides faster healing times.

Contrast agents (dye/medium) are used in balloon angioplasty techniques to improve the visibility by X ray of the apparatus with respect to the diseased area. Contrast agents are available with different viscosity and osmotic concentrations. Reduction in contrast osmolality has been linked to improved safety. Older contrast agents with an osmolality up to 8 times that of blood impose increased risk of PCI procedural complications. Newer contrast agents such as Iohexol (trade name Omnipaque™, General Electric Healthcare, General Electric Company, Chicago, Ill., United States) are either low-osmolar contrast media (LOCM, 2-3 times the osmolality of blood), or iso-osmolar contrast media (IOCM, similar osmolality with blood). [1, 2]

Another key factor in achieving successful PCI procedure is the viscosity of the contrast agent. Higher viscosity of contrast agents results in prolonged coronary balloon deflation times which could be detrimental during interventions when vessel occlusion times should be minimized. To counter the effects of high contrast viscosity on deflation, angioplasty balloon manufacturers recommend diluting contrast agent with saline fluid such as a 1:1 contrast-saline mix [3, 4, 5].

Minimally invasive balloon angioplasty and advanced techniques such as drug eluting stents (DES) and drug eluting balloons (DEB) have been successful. However, incidences of in stent restenosis (ISR) and late stent thrombosis (LST) leading to further complications is common. Another area of concern is calcified blood vessels which have shown to respond poorly to angioplasty techniques particularly in the peripheral interventions. This further increases likelihood of ISR, LST, target lesion revascularization (TLR) and vessel dissection.

CAD and PAD lesions are both systemic manifestations of atherosclerosis and their treatments including interventional methods (e.g. balloon angioplasty, stenting, atherectomy) are similar. However, the success rate in PAD interventions is lower and more costly [6]. One of the key factors is the different disease morphologies observed between PAD and CAD. Plaque concentricity and increased calcification have been reported to affect acute recoil and/or more dissections, poorer stent expansion, and poorer long-term outcome. PAD lesions treated with balloon angioplasty including DEB and DES have been reported to show poorer outcomes owing to incomplete stent expansion and more dissections due to PAD lesions featuring more calcified regions as well as concentric plaque compared to CAD lesions. The severity of lesion calcification is a predictor for late lumen loss in PAD treatment with a drug-eluting balloon [7].

A systematic review and meta-analysis of RCT (randomised clinical trials) showed an increased risk of death over the duration of 2 years onwards following application of paclitaxel (commonly used in DCB/DES) coated balloons and stents in the femoropopliteal artery of the lower limbs. This is attributed to longer half-life of the drug resulting in the late paclitaxel toxicity, microparticle formation that may embolize the downstream circulation. The concentration of paclitaxel in most drug-coated balloons and stents for the peripheral lesions is much higher than the drug eluting coronary stents. Further evidence suggests that only 1% to 10% of the paclitaxel dose gets transferred into the target vessel wall, and as much as 90% gets lost into the systemic circulation to unknown consequences [8, 9].

Lesion length in PAD can be multiple times the size of coronary lesions with PAD lesions measuring more than 250 mm [10, 11]. Longer lesions treated with balloon angioplasty techniques comprise overlapping regions requiring multiple treatments causing increased inflation time. In particular, overlapping which is a technically challenging technique is associated with an increased rate of stent thrombosis and in-stent restenosis. Overlap between bioresorbable scaffolds (BRS) is even more challenging when higher strut thickness and poor angiographic visualization of the scaffolds platform interferes with conventional interventions [12].

Stent fracture is another issue while achieving long-term patency in long SFA lesions that can result into lower patency. Lesions treated with more than one stent show significantly higher rates of multiple fractures and restenosis when compared to a single stent placement. In general, overlapping reduces the flexibility and the physical stress response of the stents leads to stent distortion and multiple fractures [13].

In cases of stenosis/occlusion more than 100 mm, a more invasive bypass surgery is preferred due to the better long-term patency rate as compared to the balloon angioplasty. Bypass surgery is the gold standard for the treatment of critically stenotic or occlusive lesions in the femoropopliteal artery (FPA). 1-year patency rate is lower when using balloon angioplasty if the length of the lesion is <100 mm. The SFA and popliteal PA when occluded are potentially compromised by high calcium content within the plaque and vessel wall, long length of lesions, and unique dynamic forces found within these arteries; therefore, surgery is still recommended as the preferred treatment modality. The diameter of the balloon is defined according to the diameter of the target arteries and be sized to reduce the risk of rupture of the targeted artery; also, the length of the balloon catheter has to include the whole lesion site [14].

Commercially available balloons for peripheral interventions range in size with diameters of 2 mm to 14 mm (Boston Sci-Charger-Abbott Armada), lengths extending to 300 mm (Medtronic Pacific Xtreme) with rated burst pressure up to 17 atm. Subsequently stent diameters range from 5 mm-10 mm with length extending up to 200 mm (Medtronic Everflex).

Lumen diameter of some of the peripheral arteries can be smaller than coronary arteries making the former more challenging to get access and place stents. External diameter of typical SFA and popliteal arteries measure about 7 mm with a lumen (internal) diameter of 6 mm. This further reduces to ˜5 mm in females, [15, 16, 17]. Internal diameter of anterior and posterior tibial arteries at the origin is about 3.5-4 mm which further gets smaller lower down the leg [18] whereas the internal diameter for coronary arteries such as left main artery can be about 5 mm [19].

In addition, due to their placement location and size, peripheral stents quite often result in restricted leg movement of the patient. As stated earlier, plaque modification may be a primary impactful factor to deliver successful and long-lasting peripheral interventions.

Several types of coaxial-based antennas, including the coaxial slot antenna coaxial monopole, dipole antenna coaxial cap-choke antennas and others, have been designed for microwave ablation [21]. However, these antennas are usually large in outer diameter making them unsuitable for angiographic procedures such as PCI scale. And although these designs have been effective for power deposition to the required area, the electromagnetic energy pattern is highly localised and non-uniform [22, 23, 24].

Previous attempts have been made relating to a multiple slot microwave antenna for vascular applications. EP 1613230 B describes a microwave antenna for cardiac ablation and a method for making such an antenna using conducting rings. A combination of the slots and rings is then used to obtain a satisfactory reflection coefficient (S11) [25]. Leaky coaxial antennas for other medical applications have been also proposed [26, 27]. However, none of them provide an apparatus and an optimising method to achieve uniform deposition of energy across a longer length of a biological tissue.

### SUMMARY

In a first aspect, there is provided a microwave system, comprising: a microwave generator; a controller configured to control the microwave generator to generate microwave energy having a selected operational frequency or range of frequencies; a microwave cable configured to deliver the microwave energy to a microwave antenna extending from or coupled to a distal end of the microwave cable; and the microwave antenna.

A property of the microwave antenna may vary along a portion of the microwave antenna. A profile of radiation emitted by the microwave antenna may be dependent on the varying property of the microwave antenna. The property may comprise at least one of slot width, slot size, slot length, conducting element width, conducting element size, conducting element length, a dielectric property, a dielectric constant, a diameter.

The variation of the property may be selected such that, when microwave energy having a selected operational frequency or range of frequencies is delivered to the microwave antenna, a desired radiation profile is emitted by the microwave antenna. The desired radiation profile may comprise substantially uniform radiation along the portion of the microwave antenna. The desired radiation profile may comprise a variation in radiated energy of no more than 20% along the portion of the microwave antenna, optionally no more than 10%, further optionally no more than 5%. The desired radiation profile may comprise a variation in radiated energy of no more than 20% along the length of the lesion to be treated, optionally no more than 10%, further optionally no more than 5%. The desired radiation profile may comprise a variation in SAR magnitude of no more than 20% along the portion of the microwave antenna, optionally no more than 10%, further optionally no more than 5%. The desired radiation profile may comprise a variation in SAR magnitude of no more than 20% along the length of the lesion to be treated, optionally no more than 10%, further optionally no more than 5%.

The microwave antenna may comprise a plurality of slots distributed along a portion of the microwave antenna. A width of the slots may vary with distance along the portion of the microwave antenna.

A width of at least one of the plurality of slots may be different from a width of a further at least one of the plurality of slots. Each of the slots may have a different width.

The width of the slots may be selected such that, when microwave energy having a selected operational frequency or range of frequencies is delivered to the microwave antenna, a desired radiation profile is emitted by the microwave antenna. The desired radiation profile comprises substantially uniform radiation along the portion of the microwave antenna.

The width of the slots may increase with distance from the generator. The width of the slots may increase such that each slot is wider than any slot that is closer to the generator.

At least some of the slots may be annular slots. All of the slots may be annular slots. At least some of the slots may be part-annular slots. All of the slots may be part-annular slots.

The cable may be a coaxial cable. The slots may be formed by removal of respective sections of an outer conductor of the coaxial cable. The slots may be formed by removal of respective sections of an outer conductor of the coaxial cable and of an outer insulating sheath of the coaxial cable.

The slots may be separated by conducting elements.

A length of the conducting elements may be selected to obtain a or the desired radiation profile.

At least some of the conducting elements may be of the same length.

A system may further comprise a balloon into which the microwave antenna is insertable.

The microwave antenna may be configured to be inserted into the balloon so that a longitudinal axis of the microwave antenna is angled relative to a longitudinal axis of the balloon. An angle of the microwave antenna relative to the longitudinal axis of the balloon may be less than 20°, optionally less than 10°, optionally less than 5°, further optionally less than 2°, further optionally less than 1°. The angle may be selected based on a condition to be treated. The angle may be selected based on an area to be treated.

The microwave antenna may be configured to be inserted into the balloon so that a longitudinal axis of the microwave antenna is offset from a longitudinal axis of the balloon.

The microwave antenna may be configured to perform tissue hyperthermia at the operational frequency or range of frequencies. The microwave antenna may be configured to perform plaque modification at the operational frequency or range of frequencies. The microwave antenna may be configured to treat cardiovascular disease.

A diameter of the microwave antenna may be selected such as to be insertable into a vessel to be treated. A diameter of the cable may be selected such as to be insertable into a vessel to be treated.

The vessel to be treated may be a peripheral vessel.

A diameter of the microwave antenna may be less than 10 mm, optionally less than 5 mm, further optionally less than 2 mm. A diameter of the cable may be less than 10 mm, optionally less than 5 mm, further optionally less than 2 mm.

The portion of the antenna may comprise at least 50 mm in length, optionally at least 100 mm, further optionally at least 200 mm.

In a further aspect, which may be provided independently, there is provided a microwave antenna comprising a plurality of slots distributed along a portion of the microwave antenna, wherein a width of the slots varies with distance along the portion of the microwave antenna, optionally wherein the widths of the slots are selected such that, when microwave energy having a selected operational frequency or range of frequencies is delivered to the microwave antenna, a desired radiation profile is emitted by the microwave antenna.

In a further method, which may be provided independently, there is provided a method of performing a tissue heating process comprising: positioning a microwave antenna in or adjacent to a treatment area, generating microwave energy by a microwave generator, the microwave energy having a selected operational frequency of range of frequencies; providing the microwave energy to the microwave antenna; and heating the treatment area by radiation of microwave energy from the microwave antenna.

A property of the microwave antenna may vary along a portion of the microwave antenna. A profile of radiation emitted by the microwave antenna may be dependent on the varying property of the microwave antenna. The property may comprise at least one of slot width, slot size, slot length, conducting element width, conducting element size, conducting element length, a dielectric property, a dielectric constant, a diameter.

The variation of the property may be selected such that, when microwave energy having a selected operational frequency or range of frequencies is delivered to the microwave antenna, a desired radiation profile is emitted by the microwave antenna. The desired radiation profile may comprise substantially uniform radiation along the portion of the microwave antenna. The desired radiation profile may comprise a variation in radiated energy of no more than 20% along the portion of the microwave antenna, optionally no more than 10%, further optionally no more than 5%.

The microwave antenna may comprise a plurality of slots distributed along a portion of the microwave antenna. A width of the slots may vary with distance along the portion of the microwave antenna.

Positioning the microwave antenna in or adjacent to the treatment area may comprise inserting the microwave antenna into a balloon and inserting the balloon into a vessel.

The vessel may be a peripheral vessel.

The widths of the slots may be selected such that, when microwave energy having a selected operational frequency or range of frequencies is delivered to the antenna, a desired radiation profile is emitted by the microwave antenna into the treatment area.

The desired radiation profile may comprise substantially uniform radiation along the portion of the microwave antenna.

The treatment area may comprise a lesion.

The tissue heating may be such as to cause tissue hyperthermia in the treatment area.

The tissue heating may be such as to cause plaque modification in the treatment area.

In a further aspect, which may be provided independently, there is provided a method for designing a microwave antenna, the method comprising: a) simulating radiation from an initial antenna design, the initial antenna design comprising a plurality of slots arranged along a portion of a coaxial cable; b) fitting the simulated radiation to an attenuation curve; c) using an inverse of the attenuation curve to determine a respective slot width for each of the plurality of slots; and d) designing a microwave antenna having the determined slot widths.

The slot widths may be such as to provide a desired radiation profile when microwave energy is supplied to the antenna.

The desired radiation profile may comprise substantially uniform radiation along the portion of the microwave antenna.

The plurality of slots of the initial antenna design may be equally sized. The plurality of slots of the initial antenna design may be equally spaced along the coaxial cable.

The simulating of the radiation may comprise simulating radiation into a predetermined material having a known relative permittivity.

The predetermined material may comprise saline. The predetermined material may comprise a contrast material. The predetermined material may comprise a combination of saline and contrast material. The predetermined material may comprise deionised water or RO (reverse osmosis purified) water, and/or for example material providing high dielectric constant (relative permittivity) and/or a low or zero loss tangent and/or low electrical conductivity. The predetermined material may comprise a combination of contrast material and deionised water and/or RO water and/or saline.

The plurality of slots may be separated by conducting elements. Each conducting element may be of the same size. Each conducting element may be of the same length. The method may further comprise determining a size of the conducting elements based on the relative permittivity and the antenna length.

The designing of the microwave antenna may comprise evaluating an effective wavelength in the material. The effective wavelength may be evaluated using a frequency of operation of the antenna and the relative permittivity. The length of the conducting elements may be determined to be a product of the effective wavelength and a multiplying factor. The multiplying factor may be dependent on the antenna length. The multiplying factor may be dependent on the effective wavelength. The multiplying factor may be dependent on a diameter of the coaxial cable. The multiplying factor may be dependent on a size of a treatment area to be treated. The multiplying factor may be dependent on a length of a lesion to be treated.

Using an inverse of the attenuation curve to determine a respective slot width for each of the plurality of slots may comprise determining a leakage factor function from the attenuation curve and using the leakage factor function to determine the slot widths.

The method may further comprise iteratively repeating steps a) to c) until a desired radiation pattern is achieved.

In a further aspect, which may be provided independently, there is provided a microwave antenna designed using a method as described or claimed herein.

In a further aspect, which may be provided independently there is provided a system as described or claimed herein, wherein the microwave applicator is designed using a method as described or claimed herein.

In a further aspect, which may be provided independently, there is provided a method of fabricating a microwave antenna, the method comprising: providing a coaxial cable; and at a distal end of the coaxial cable, selectively removing a plurality of sections of an outer conductor of the coaxial cable to expose sections of the inner conductor, thereby forming a plurality of radiating slots, wherein slot widths of the radiating slots are determined using a method as described or claimed herein. The method may further comprise selectively removing a plurality of sections of an outer insulating sheath of the coaxial cable.

The sections removed from the outer conductor may be annular sections. The sections removed from the outer insulating sheath may be annular sections. The slots may be annular slots.

A minimally invasive treatment that is able to treat a long lesion primarily by plaque modification with the benefits of reduced inflation time and minimised use of overlapping regions may be provided. There may be provided a method and system for treating conditions such as but not limited to peripheral vascular disease (PVD) using microwave energy system.

A microwave system for providing local hyperthermia comprises a microwave antenna of a millimetric diameter capable of providing controlled hyperthermia uniformly over the entire length of a long lesion (e.g. greater than 50 mm). The microwave radiation is limited to the walls of the vessel and is restricted from travelling further into the muscle tissue.

In accordance with a further aspect, which may be provided independently, there is provided a microwave system, comprising:
a microwave generator;
a microwave cable;
a microwave applicator for delivering microwave radiation generated by the microwave generator in the peripheral blood vessels.

The generated microwave radiation may comprise a series of pulsed microwave signals. One or more pulse parameters of the generated microwave radiation may comprise at least one of: a pulse width, a pulse frequency, a pulse height, a pulse duration. The modulated microwave signals may be modulated in accordance with a modulation scheme to vary the average power delivered. The modulation scheme may comprise at least one of: amplitude modulation pulsing, pulse-width modulation and/or on off keying. The output may be modulated by control of linear gain to create a variable amplitude control of power.

The microwave generator may be configured to delivery microwave radiation comprising a frequency between 900 MHz and 30 GHz, optionally wherein the frequency is about 915 MHz, about 2.45 GHz, about 5.8 GHz, about 8.0 GHz, or about 24.125 GHz.

The generated microwave power may range from 0.1W to 100W, optionally between 1W to 20W. The treatment time may range from one thousandth of a second to 1 s and 1 s to 1800 s, optionally between 1 s and 10 s.

The microwave generator may be configured to deliver one of microwave radiation at a fixed frequency, microwave radiation at a variable frequency and/or modulated microwave radiation.

The microwave applicator may comprise a radiating antenna that is capable of providing uniform and controlled hyperthermia by microwave radiation to the biological tissue over a long length through minimally invasive techniques such as PCI.

Design parameters of said microwave antenna may be derived using a method as claimed or described herein.

There may be provided a method or system substantially as described herein with reference to the accompanying drawings.

Features in one aspect may be provided as features in any other aspect as appropriate. For example, features of a method may be provided as features of an apparatus and vice versa. Any feature or features in one aspect may be provided in combination with any suitable feature or features in any other aspect.

### BRIEF DESCRIPTION OF THE DRAWINGS

Embodiments of the invention are shown in the following figures:

FIG. 1 is a schematic illustration of a microwave treatment system, in accordance with embodiments;

FIG. 2 is an illustration of the FEA (finite element analysis) simulation model depicting microwave antenna of the microwave applicator assembly placed in the blood vessel, in accordance with embodiments;

FIG. 3 is a cross-sectional view of the microwave antenna of the microwave applicator assembly placed in the blood vessel, in accordance with embodiments;

FIG. 4 is a detailed view of the microwave antenna of the microwave applicator assembly placed in the blood vessel, in accordance with embodiments;

FIG. 5 illustrates a typical performance of a nine-slot microwave antenna with equal slot widths loaded in saline showing non-uniform SAR distribution along the blood vessel;

FIG. 6 demonstrates a manual method of achieving uniform distribution of microwave energy in the blood vessel using present invention;

FIG. 7 represents power propagation along an example nine-slot antenna with equal slot widths, in accordance with embodiments;

FIG. 8 represents an inverse attenuation fit to evaluate optimum slot width in order to achieve balanced uniform radiation across all the slot elements;

FIG. 9 illustrates uniform SAR distribution along the blood vessel using a nine-slot microwave antenna loaded in saline designed and optimised using the present invention;

FIG. 10 illustrates S11 plot of a nine-slot microwave antenna loaded in saline and radiated into a blood vessel;

FIG. 11 illustrates uniform SAR distribution along the blood vessel using a twelve-slot microwave antenna designed using the present invention loaded in saline;

FIG. 12 illustrates S11 plot of a twelve-slot microwave antenna loaded in saline and radiated into a blood vessel;

FIG. 13 illustrates uniform SAR distribution along the blood vessel using a nine-slot microwave antenna loaded in (1:1) solution of Omnipaque™ and saline;

FIG. 14 illustrates S11 plot of the nine-slot microwave antenna loaded in (1:1) solution of Omnipaque™ and saline and radiated into blood vessel;

FIG. 15 depicts a method of achieving preferential unilateral and uniform distribution of microwave energy in the blood vessel in accordance with an embodiment;

FIG. 16 shows a prototype microwave antenna manufactured based on the present invention, in accordance with embodiments;

FIG. 17 illustrates S11 plot of the prototype microwave antenna measure using a VNA when loaded into the air, in accordance with embodiments;

FIG. 18 illustrates S11 plot of the prototype microwave antenna measured using a VNA when loaded into saline, in accordance with embodiments;

FIG. 19 shows a typical radiation pattern for and ex-vivo tissue achieved using the present invention, in accordance with embodiments.

### DETAILED DESCRIPTION OF THE DRAWINGS

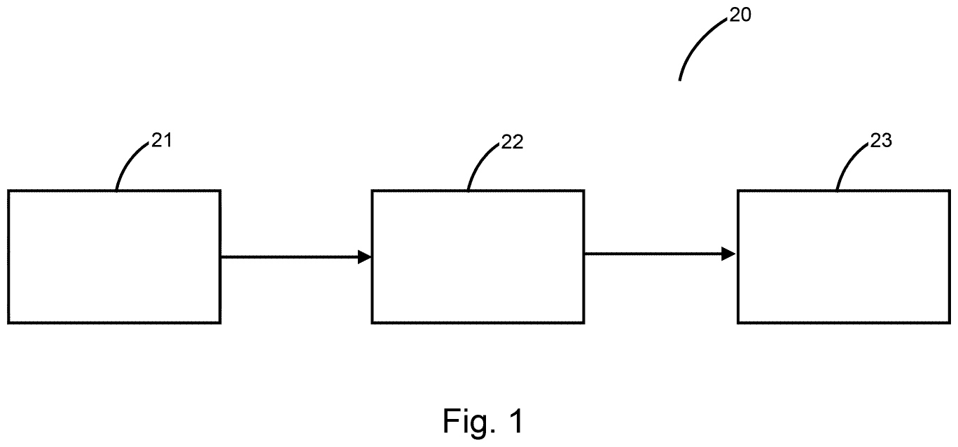

A microwave radiation delivery system 20, in accordance with embodiments, for treating a biological tissue, is illustrated in FIG. 1. The system comprises a microwave generator 21 for generating microwave radiation, a flexible or rigid interconnecting cable 22 and a microwave applicator assembly 23 for delivering microwave radiation to a biological tissue. Microwave applicator assembly 23 may also be simply referred to as a microwave applicator or microwave antenna in this document. The microwave radiation delivery system may further comprise a controller (not shown) which is configured to select an operational frequency or range of frequencies to be supplied by the generator. A frequency of the microwave radiation supplied by the microwave generator 21 may be between 900 MHz and 30 GHz, for example about 915 MHz, about 2.45 GHz, about 5.8 GHz, about 8.0 GHz, or about 24.125 GHz. In some embodiments, the microwave radiation supplied is pulsed.

In use, the microwave applicator assembly is introduced into a balloon. The balloon may be an angioplasty balloon as described above. The balloon may have a length that corresponds to a length of lesion to be treated, for example greater than 50 mm, greater than 100 mm, or greater than 200 mm. The balloon may have a diameter that corresponds to a size of blood vessel to be treated. For example, the lesion is in a peripheral vessel, the vessel may be small. An inflated diameter of the balloon may be less than 15 mm, optionally less than 10 mm, optionally less than 7 mm, further optionally less than 5 mm.

The balloon is introduced into a blood vessel, for example a peripheral vessel in which a lesion has been identified. When in position at the lesion, the balloon is inflated using an inflation fluid, for example saline or a mix of a contrast material and saline. Microwave radiation is supplied to the microwave applicator assembly and is radiated from the microwave antenna assembly through the inflated balloon and into surrounding vessel tissue. The microwave radiation is used to treat the surrounding tissue, for example to perform plaque modification of the lesion at which the balloon is situated.

For the purpose of demonstration, testing and validation, simulation models presented in this document are designed using saline on its own and a 1:1 mixture of Omnipaque™ and saline (Omnipaque™ is a widely used solution for angiography and angioplasty techniques; referred as contrast solution henceforth in this document). Alternatively, other solutions and solutions with different ratio of saline and contrast dye can be used. Dielectric properties (relative permittivity, loss tangent and electrical conductivity) of saline and contrast solution were measured in-house using “DAK3.5: 200 MHz-20 GHz—Dielectric measurement package” apparatus by SPEAG (Zurich, Switzerland).

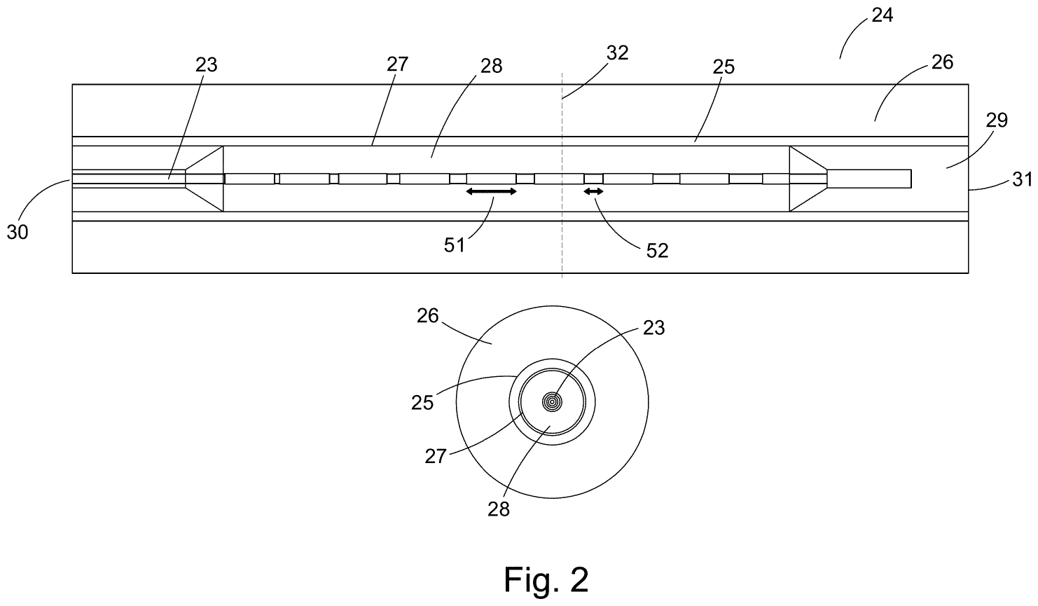

FIG. 2 represents a FEA simulation model assembly 24 constructed and simulated in HFSS (High Frequency Structural Simulator, ANSYS Inc. Canonsburg, USA) depicting microwave antenna 23 placed in a blood vessel 25 surrounded by blood 29 and muscle tissue 26, in accordance with embodiments. An inflated balloon 27 is filled with a solution of either saline, contrast solution, deionized water or mix of two or more thereof 28. In use, the antenna 23 is placed inside in the inflated balloon 27 thus preventing direct contact between the antenna 23 and blood vessel 25. The antenna 23 is introduced from proximal end 30 to the distal end 31, where the proximal end 30 of the antenna 23 is the end that is coupled to the cable 22. Antenna 23 has key geometrical parameters such as conductor length 51 and slot width 52. The geometrical parameters of the antenna 23 are described in greater detail below.

FIG. 2 also includes a cross sectional view of the antenna 23, which is shown through the section line 32.

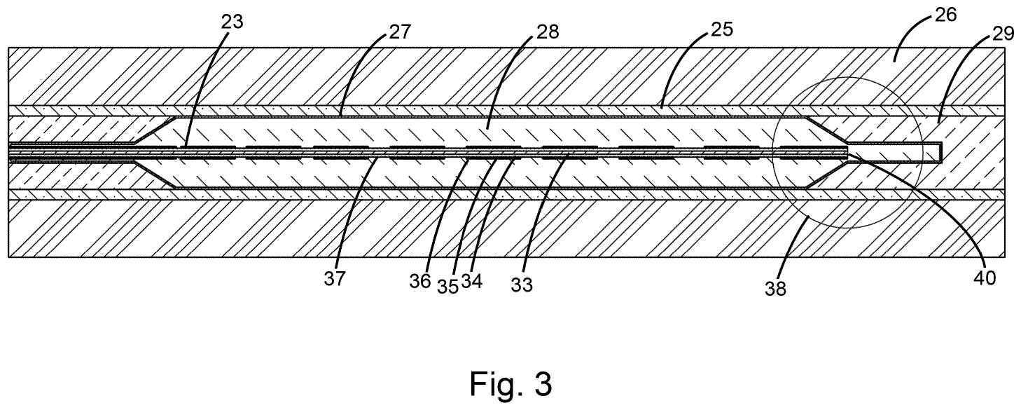

A cross-section view of FIG. 2 is illustrated in FIG. 3. The coaxial antenna components of the microwave antenna 23 are metal inner conductor 33 and outer conductor 35 separated by an insulating material 34 such as dielectric PTFE, polyethylene, Teflon or another polymer. In other embodiments, any suitable insulating material may be used. An outer insulating sheath 36 of an inert polymer such as FEP may be present. In other embodiments, any suitable outer sheath material may be used.

In other embodiments, the antenna 23 may comprise additional components. In further embodiments, one or more of the components of the embodiment of FIGS. 2 and 3 may be removed or replaced with one or more alternative components.

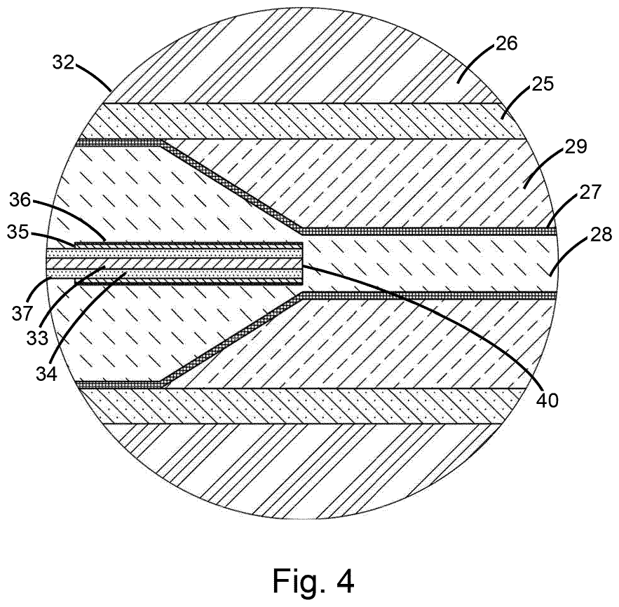

To produce the antenna 23 of FIGS. 2 and 3, a plurality of annular slots of the antenna 23 are formed in a coaxial cable. A simple radiating annular slot such as 37 with a specific slot width is constructed by removing sections of outer conductor 35 and outer insulating sheath 36 thus exposing the inner dielectric layer 34. Distal end 40 of the antenna 23 may be terminated with a metal interface. The metal interface 40 may be soldered, affixed using epoxy or applied using any other relevant method. A detail view 32 of the section is depicted in FIG. 4 highlighting components of the model assembly shown in FIGS. 2 and 3.

In further embodiments, any suitable shape of slot may be used. For example, the slot may be part-annular and may not extend around the entire circumference of the coaxial cable.

The microwave antenna 23 connected to a microwave generator through a microwave cable comprises non-uniform annular multiple slots varying in size. Multiple slots of the antenna 23, when combined, behave as an array which collectively affect the performance of the antenna. A phase difference between the slots, proportional to the distance between the slots relative to the wavelength in the dielectric, causes constructive and destructive interference [20]. Thus, designing and constructing such antenna over a longer length is often challenging.

The antenna performance required for treating long lesions uniformly may depend on various factors such as geometrical design parameters of the antenna, length of the antenna, length of the lesion to be treated, dielectric properties of the materials used, effective wavelength (λ_{eff}) of the target material, frequency of operation, cable diameter etc. These factors influence antenna performance simultaneously by changing the attenuation constant (α), phase constant (β) and hence the propagation constant (γ) throughout the antenna (see equation 2 below). It has been found that finding optimised parameters to achieve the required results may be quite tedious. For example, design methods may comprise running long parametric analysis for each variation.

Embodiments described herein may provide a method in which by keeping the known parameter values constant such as length of the lesion to be treated, dielectric properties of the materials used, frequency of operation, cable diameter, slot width and position along the antenna can be derived to achieve uniform radiation emission across the whole long lesion length using a derived function.

In the present embodiment, the length of lesion and the selected operational frequency or range of frequencies are known. The cable diameter and the dielectric properties of the materials used to form the antenna are known and remain constant along the length of the antenna. All of the conductive rings between the slots have the same length which is selected as part of the antenna design process. The slot width is varied along the length of the antenna to obtain a desired radiation pattern.

In other embodiments, any suitable parameters may be kept constant, and any suitable further parameters may be varied in order to obtain a desired radiation pattern. For example, a cable diameter, dielectric property, slot width, slot size, slot shape, conductive element width, conductive element size and/or conductive element shape may be varied along the length of the antenna to obtain the desired radiation pattern.

The effective wavelength (λ_{eff}) of the material the antenna is radiated into is calculated from equation 1.

$$λ eff = \frac{c}{f √ ϵ r} \qquad Equation 1$$

where c is the speed of light in free space (m/s), f is the operating frequency of the microwave generator (for example 8 GHz), and ϵr is the relative permittivity of the material the antenna is radiated into [21]. The material that the antenna is radiated into may be, for example, saline, deionised water, reverse osmosis (RO) water or a mixture of contrast material and any one or more of saline, deionised water, RO water.

The width of each slot influences the performance of the coaxial slot antenna. The slot width (FIG. 2, 52) depends on various factors for example, the number of slots used, λ_{eff} of the material and the spacing between each slot (termed as “conductor length”, FIG. 2, 51) etc.

It is known that a narrower slot radiates less energy than a wider slot. However, over the length of the antenna, slots at the proximal end of the antenna would radiate more energy than the slots towards the distal end, where proximal refers to near end of the instrument to the operator and distal refers to the far end away from the operator—generally in the patient.

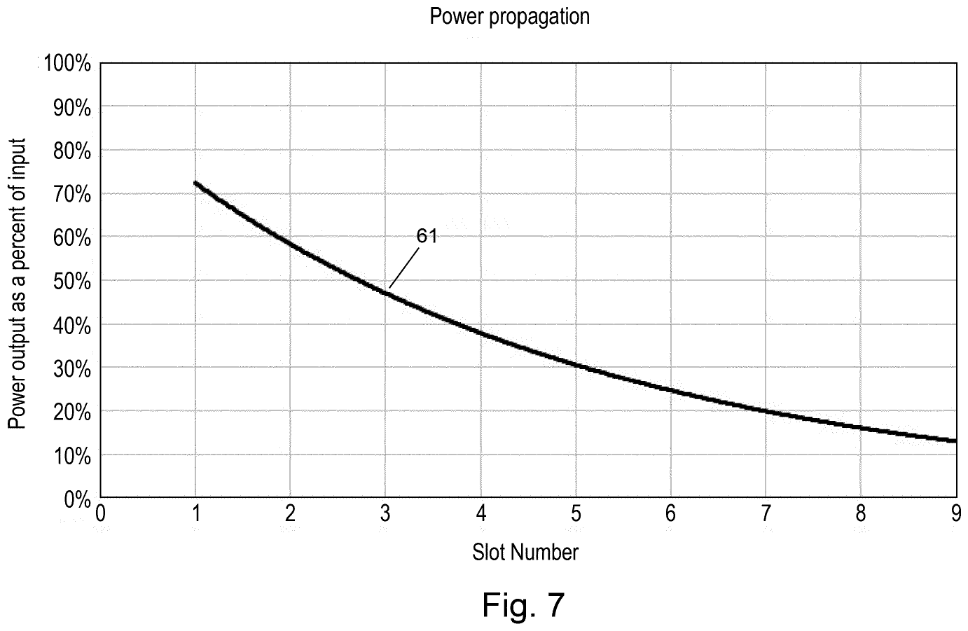

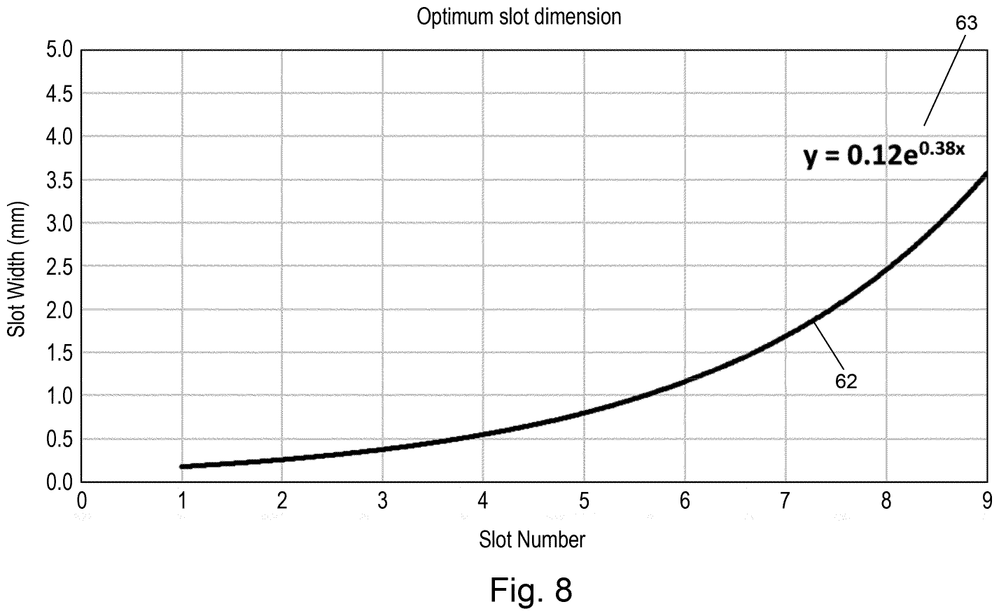

For example, as shown in FIG. 7 and FIG. 8 below, in a typical multi-slot antenna 50 (nine-slot antenna for this instance) comprising slots of equal widths, the input power decays as an exponential function of “leakage factor” along the axis of propagation. In other words, an attenuation constant increases from the beginning (proximal end) of the antenna towards the end (distal end). This adds further complexity to achieving optimised parameters of the design.

The propagation constant y in equation 2 has two components: a, attenuation constant (real part) and β, phase constant (imaginary part). The attenuation constant decreases the signal amplitude along a transmission line whereas the phase constant determines phase of the signal along a transmission line.

Equation 2

γ=α+jβ (2)

λ_{eff} can be evaluated using equation 1. The conductor length 51 as shown in FIG. 2 is the uniform periodic distance between each slot. The conductor length is calculated as a product of λ_{eff} and a multiplying factor. The multiplying factor is evaluated using equation 3. For example, when using saline and the contrast solution as a target medium, following equation (3) can be used:

Equation 3

m=1.9-0.0015*a*I (3)

Where m=multiplying factor, a=antenna length, I=λ_{eff}. It should be noted that equation 3 fits for the current combination of design parameters such as cable diameter, frequency of operation, lesion length, antenna length, target medium etc. and would have a different form when any one or all the parameters are different.

The performance of a microwave antenna in this description is primarily depicted using return loss or S11 plot. The S11 parameter represents how much power is reflected from the antenna back to the input port. It can also be termed as the reflection coefficient or return loss. Thus, for example, an antenna S11=0 dB implies all the power is reflected from the antenna and nothing is radiated. S11=−3 dB indicates 50% power being reflected and 50% being delivered to the antenna i.e. either radiated or absorbed as losses within the antenna. Typically, antennas are designed to be low loss. S11=−10dB indicates 10% power being reflected and 90% delivered.

Another key parameter for analysing microwave antenna performance in particular involving biological tissues is SAR (Surface Absorption rate). SAR is the amount of electromagnetic radiation that is absorbed by the human body.

$$SAR = \frac{P}{ρ} = \frac{σ {\vert E \vert}^{2}}{ρ} \qquad Equation 4$$

Where, P is the power [watts] absorbed in the tissue and p is the mass density of the medium [Kg/m³], |E| is the rms magnitude of the electric field strength vector [V/m] and σ is the electrical conductivity of the tissue. SAR is correlated to the temperature gradient in tissue thus is a key parameter while evaluating efficiency and safety of any EM based treatment. In terms of the temperature gradient (ΔT) induced in the system, SAR can be represented as:

$$SAR = C \frac{Δ T}{Δ t} \qquad Equation 5$$

Where, C is the specific heat [J/kgK] and At is treatment time [s]. Thus, the temperature [° C. or ° K] rise ΔT in the biological tissue (also known as hyperthermia) by absorbed EM energy can be evaluated using SAR [22]. Antenna performances related in this invention in accordance with embodiments are elucidated using S11 (dB) and SAR plots.

Example microwave antennas in accordance with embodiments presented in this description are designed to operate at high frequency such as 8 GHz. However, antennas of similar design can be used at any microwave frequency.

Skin depth δ (m) of the waves is inversely proportional to the operating frequency f (equation 6) [23]. Therefore, working at higher frequencies provides controlled and less penetrating radiation along the surface of the tissue. The amount of radiation entering into tissues surrounded by the diseased tissue (or the tissue being treated) may be limited. In equation 6, p is the resistivity of the material (Ωm) and μ is the permeability of the material.

$$Skin depth δ = √ ( \frac{ρ}{π * μ * f} ) \qquad Equation 6$$

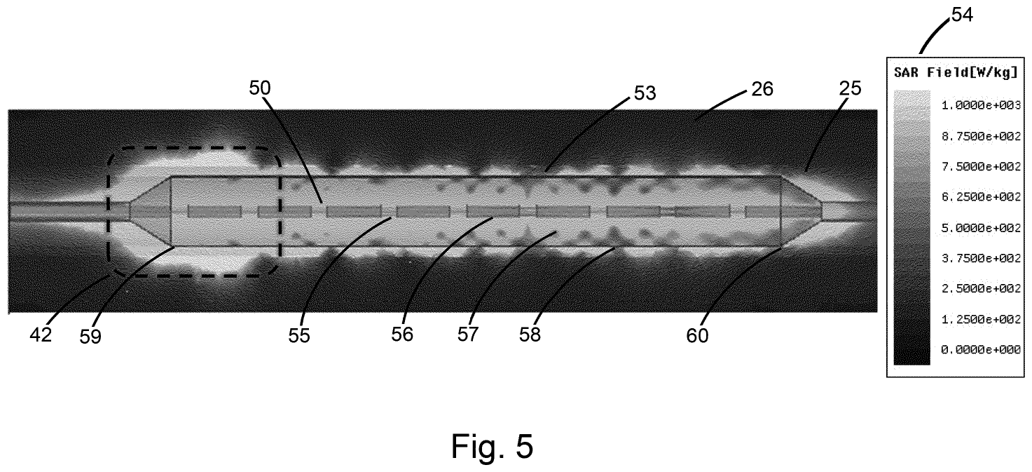

Turning back to the figures, FIG. 5 demonstrates a typical example of a non-uniform SAR distribution using a multi-slot antenna of equal slot width. The antenna 50 comprises nine slots 55 such of equal slot width and uniform conductor length 56. The antenna 50 is loaded into saline 57 and is placed in a balloon 58 of 60 mm length to treat a 60 mm lesion. Throughout this document, balloon length refers to a distance between starting 59 and ending point 60 of the main body of the balloon.

FIG. 5 shows a simulated SAR distribution from the antenna 50. Slots at the proximal end show a high deposition of SAR 42 in the blood vessel 25, extending further into the muscle tissue 26. Slots at the distal end show a lower deposition of SAR. Moreover, irregular zones of high energy 42 and low energy or null zones 53 can be seen along the length of the tissue.

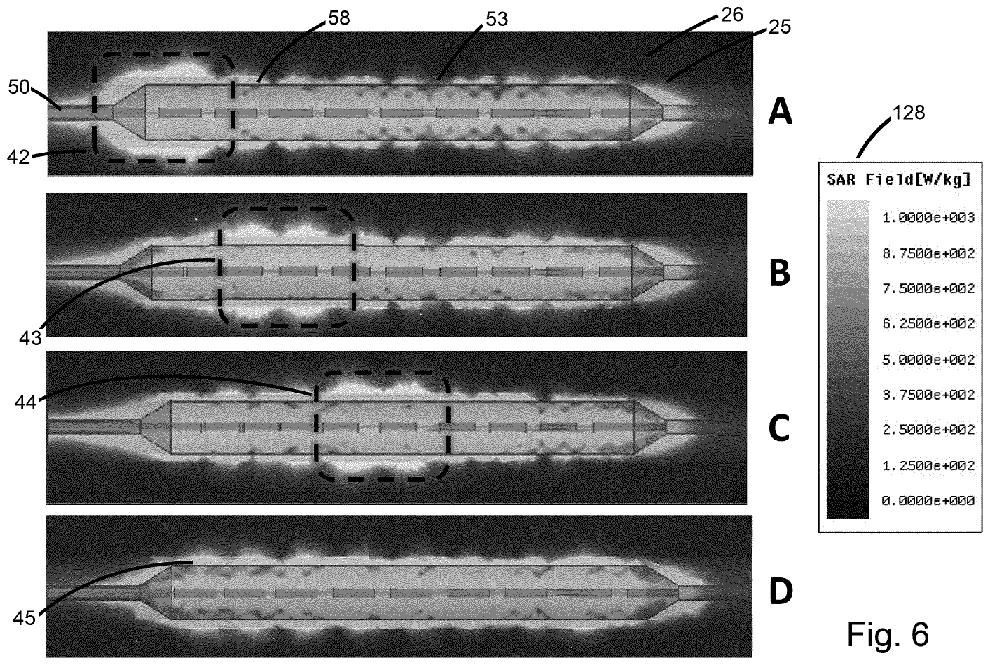

The uniform distribution of energy in a vessel may be achieved using a manual method in which one or more parameters of the antenna can be optimised as demonstrated in FIG. 6. The model assembly is similar to the FIGS. 3 and 4. A microwave antenna 50 (same as FIG. 5) with nine slots of equal slot width is simulated. In the simulation, the antenna 50 is placed in a balloon 58 to provide hyperthermia to a vessel 25 that is embedded in muscle 26. Parameters such as incorrect slot width, slot position and conductor length etc. may result in non-uniform energy depositions causing regions with high energy 42 and very low energy or null zones 53.

The model is then changed manually to adjust the distribution of energy. Regions with high energy can be moved along the antenna by either changing slot width, positions, or conductor length between each slot and other parameters. For example, the high energy zones 42, 43 and 44 may be moved towards the distal end by changing slot width and slot position only. Finally, by adjusting and balancing all these features, uniform energy distribution 45 may be achieved.

However, a manual approach may be time consuming to iteratively refine parameters to achieve the uniform performance.

Alternatively, a method of an embodiment may be applied to an antenna in order to achieve a uniform SAR distribution along the length of the lesion in the blood vessel. To achieve this, key parameters such as λ_{eff}, multiplying factor and the conductor length are first evaluated.

A generic method as follows may be provided to design a multi-slot antenna for achieving uniform radiation along the antenna of any length. The method may comprise the following steps:
1. Identify constant known factors such length of the lesion to be treated, dielectric properties of the materials used, frequency of operation, cable diameter etc.
2. Evaluate λ_{eff} in the target medium
3. Determine length of the antenna based on the known parameters.
4. Determine number of slots required.
5. Evaluate multiplying factor to identify the conductor length.
6. Using simulated results calculate the attenuation constant, α.
7. Evaluate the leakage factor from attenuation constant.
8. Apply the leakage factor function to the slot width to produce an inverse attenuation constant fit (for example FIG. 8) to balance the radiation across all the slot elements i.e. to evaluate optimum slot dimensions to create uniform energy radiation.
9. Repeat 6-8 iteratively until desired pattern is achieved.

FIG. 7 shows that in a typical multi-slot antenna 50 (nine-slot antenna for this instance) comprising slots of equal widths, the input power decays as an exponential function of a leakage factor along the axis of propagation. Power propagation along the antenna 50 is shown in FIG. 7. FIG. 8 shows a leakage factor function which increases along the length of the antenna 50. The leakage factor function may be considered to be inverse to the attenuation.

At stage 8 of the process listed above, the leakage factor function is applied to the slow width. For example, a leakage factor function such as y=0.12e^{0.38x} (y=slot width, x=slot number) shown in FIG. 8 is applied to a particular nine-slot antenna 50 with equal slot widths.

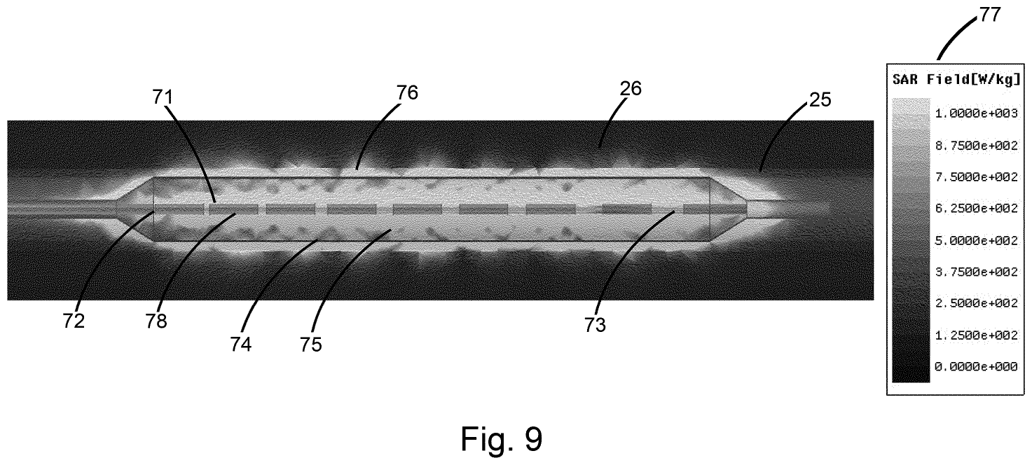

FIG. 9 shows an antenna 71 produced using the method listed above. The resultant antenna 71 produced after applying said method comprises non-uniform slots with gradually increasing slot widths from proximal to distal end. The leakage factor function may be different for other designs depending on but not limited to coaxial cable, number of slots, frequency of operation, target medium, length of lesions, length of antenna etc. For example, antenna 71 shown in FIG. 9 is designed to treat lesions up to 60 mm in length and is simulated into neat saline as the target medium. The conductor length 78 in the antenna is uniform and is correlated to λ_{eff} as 1.25*λ_{eff} (1.25 as a “multiplying factor”) and is specific for this antenna. The multiplying factor and hence the conductor length varies with λ_{eff} and material Er. For example, contrast solution comprising saline and contrast agent at 1:1 ratio has a lower Er than neat saline.

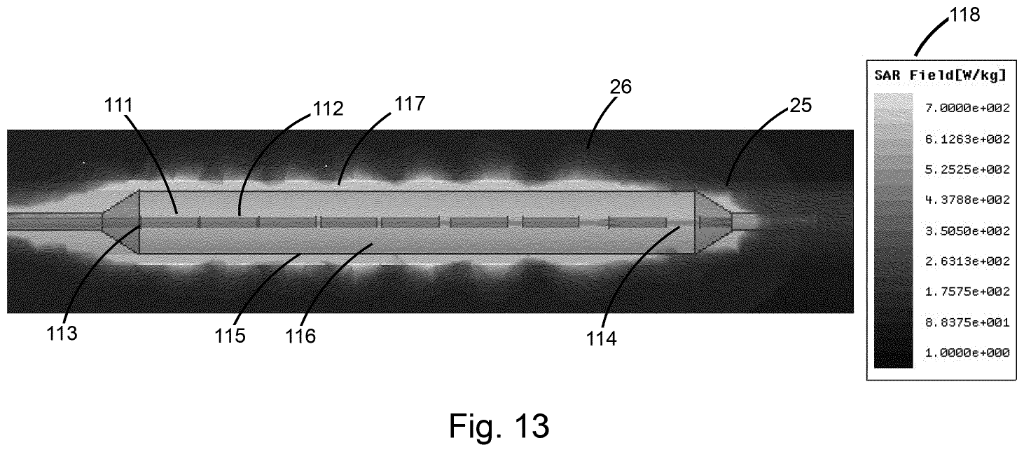

FIG. 13 illustrates a nine-slot antenna 111 simulated into contrast solution as opposed to a corresponding nine-slot antenna 71 simulated in neat saline. The uniform conductor length 112 for antenna 111 is changed to 1.15*λ_{eff} due to change in material Er.

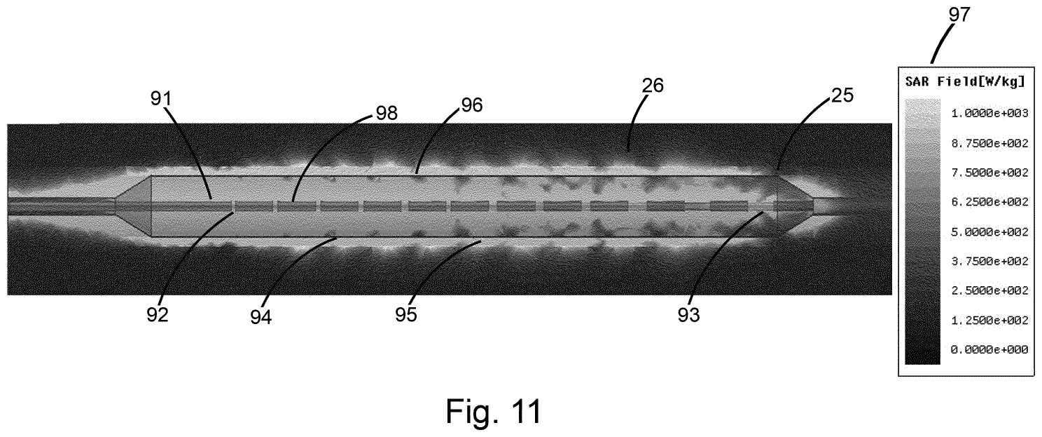

Further, when the lesion length is longer, an antenna with more slot elements may be utilised and may optimised using the method described above. For example, in FIG. 11, a twelve-slot antenna 91 simulated in saline is shown that is capable of treating lesions up to 70 mm. Antenna 91 corresponds to a nine-slot antenna 71 shown in FIG. 9 but has a shorter 98 conductor length 1*λ_{eff}. Similarly, slot width increases from proximal to distal end and is calculated using an inverse attenuation constant fit method described above.

Saline on its own is used as an example to demonstrate that the present invention can be applied for any given target medium. The dielectric properties of saline and the contrast solution were measured in-house using SPEAG dielectric apparatus.

The inflation fluid may be chosen as material with suitable values of permittivity and/or loss tangent and/or electrical conductivity (e.g. relative high relative permittivity and low loss tangent and/or low electrical conductivity) for example, DI (deionised) water and/or RO (reverse osmosis) water. The inflation fluid may be chosen as a mixture of contrast material and DI (deionised) water and/or a mixture of contrast material and RO (reverse osmosis) water. This facilitates increasing the temperature of the target tissue with dielectric heating and microwave radiation but minimising the temperature rise in the fluid restricting the heat transfer in the fluid to the heat conduction from the microwave heated tissue. This feature is particularly useful when the treatment time is longer such as longer than 1 minute. A conducting fluid may heat up rapidly and can form excess steam in the balloon which can be overcome using a fluid with low or null electrical loss.

Previous attempts have been made relating to a multiple slot microwave antenna for vascular applications. However, none of them provide an apparatus and an optimising method to achieve uniform deposition of energy across a longer length of a biological tissue as described above. Moreover, the method described above may provide a system and method of designing and constructing a microwave antenna of any diameter including millimetre scale coaxial cables to provide uniform radiation distribution across a length of lesion by knowing parameters such as lesion length to be treated, dielectric properties of the materials, cable diameter and operating frequency. For example, when treating a 70 mm lesion with a coax outer diameter of 1.2 mm, contrast solution (Er=30, a (electrical conductivity)=8S/m) when operating at a frequency, f=8 GHz: a balloon of 70 mm can be used with an antenna consisting nine slots of non-uniform increasing slot widths. The conductor length and slot width may be evaluated using a method as described above based on the known parameters and calculated multiplying factor and the leakage factor. Finally, an iterative inverse attenuation constant fit is applied to balance the radiation propagation across all slot elements and create uniform energy radiation. Using this method, the design process of an antenna to uniformly treat a lesion of any length may be optimised by algorithm.

Evaluation of slot widths is summarised in FIG. 7. An example of a typical power propagation 61 as a percentage of input power along the length of a nine-slot antenna 50 with equal slot widths is demonstrated. Slots at the proximal end of the antenna radiate more energy than the slots at the distal end resulting in an exponential reduction in the power output from slot one to slot nine and in turn providing non-uniform radiation along the length of the antenna. In order to achieve a balanced radiation through each slot element, this power decay can be compensated using a leakage factor that is evaluated from the attenuation constant. An inverse attenuation fit 62 as illustrated in FIG. 8 is then iteratively derived and applied to evaluate slot width for each slot (in mm) to achieve balanced and uniform radiation through each slot. The exponential reverse attenuation fitted with 63 an equation y=0.12e⁰³⁸x (where y=slot width, x=slot number) applies to this particular example only and would change depending on but not limited to coaxial cable, frequency of operation, target medium, length of lesions etc. Using this method, antenna 71 as shown in FIG. 9 is designed to achieve uniform distribution along the length of the lesion.

FIG. 9 illustrates a SAR plot for an example microwave antenna 71 with nine annular slots that is designed using the method presented above. A microwave antenna 71 is introduced through a 60 mm long balloon 74 and into saline fluid 75. The microwave antenna 71 emits uniform and controlled microwave radiation 76 into the vessel 25. Antenna 71 comprises nine annular slots of non-uniform width. The width for each slot is calculated by evaluating leakage factor function and by applying reverse attenuation fit as demonstrated in FIG. 7 and FIG. 8. Slots are separated by equal distance of 1.25*λ_{eff}, also referred as conductor length 78. A greyscale contour 77 of the SAR plot of FIG. 9 indicates absorbed radiation into the materials showing uniform and controlled microwave radiation 76 into the vessel 25. Radiation entering into muscle tissue 26 may be limited.

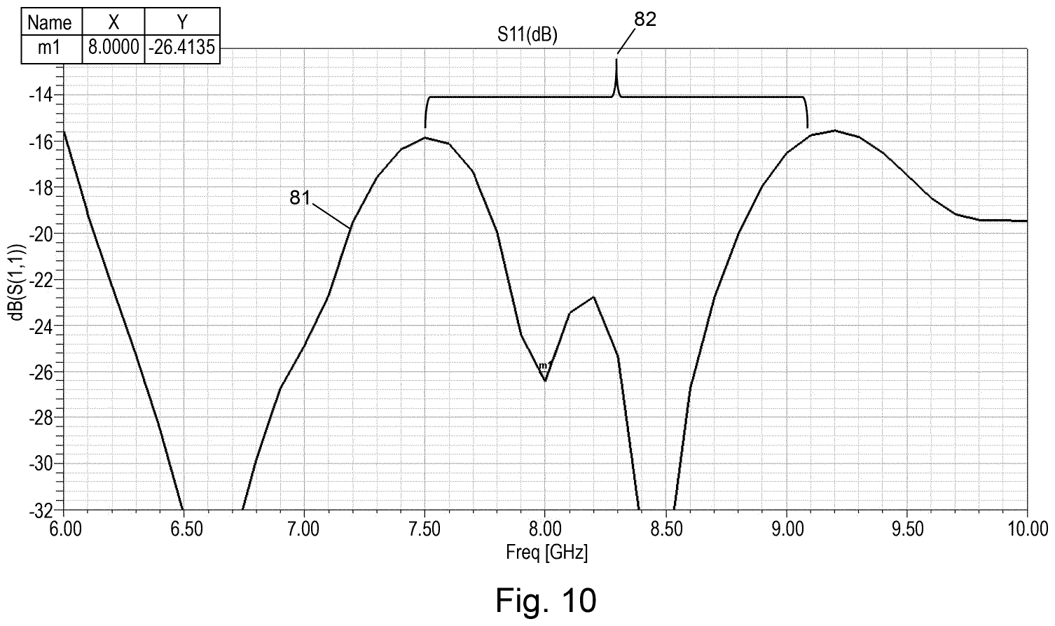

The antenna 71 is composed of nine annular slots. In other embodiments, slot widths may vary but usually increase from the proximal to distal end. i.e. from first slot 72 to the final slot (no. nine) 73. The S11 (dB) plot 81 of the antenna 71 is illustrated in FIG. 10. The antenna is tuned to radiate at 8 GHz indicating that the performance highly efficient in magnitude of −26.41 dB with a substantially wide bandwidth 82.

Longer antennas similar to FIG. 9 can be designed using the same principle presented above. A microwave antenna 91 designed to treat a lesion of to 70 mm is shown in FIG. 11. The antenna 91 is introduced through a 70 mm long balloon 94 in the saline fluid 95. The antenna 91 comprises gradually increasing twelve annular slots starting from 92 and ending at 93. With an increased length of the lesion i.e. while transitioning from a shorter antenna to a longer antenna, conductor length may generally be reduced.

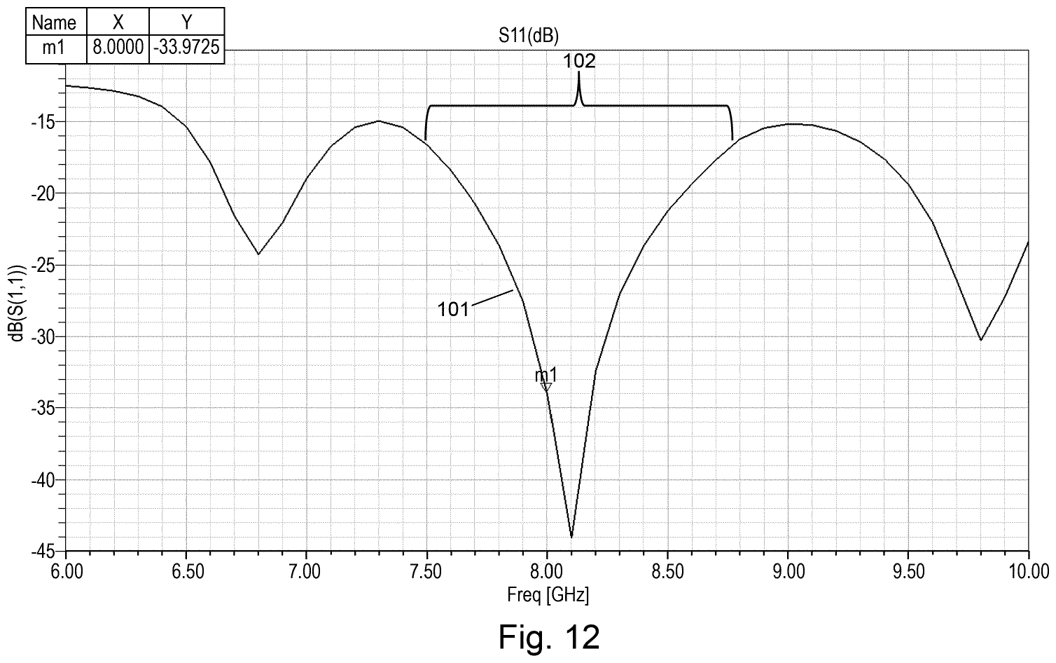

Similar to the system shown in FIG. 9, conductor length 98 for antenna 91 between each slot is uniform. However, the conductor length 98 for antenna 91 is smaller (1*λ_{eff}) than the conductor length for the nine-slot antenna (1.25*λ_{eff}). Uniform and controlled radiation 96 into the vessel 25 is demonstrated using the SAR plot 97. In FIG. 12, S11 (dB) plot 101 shows the finely tuned antenna operating at 8 GHz with efficient magnitude −33.97 dB and wide bandwidth 102.

FIG. 13 illustrates a nine-slot microwave antenna 111 to treat lesions of 60 mm introduced into the balloon 115 radiated into the vessel 25. The antenna 111 is different to the antenna 71 described in FIG. 9 which is simulated in saline fluid only. Antenna 111 in the FIG. 13 is simulated in a fluid 116 comprising contrast solution (1:1) solution of Omnipaque™ and saline. The antenna 111 comprises non-uniform increasing slots from 113 to 114. The Er (relative permittivity) of the contrast solution is lower than neat saline hence λ_{eff} for a contrast solution is higher than saline (from equation 1). Thus, to balance the interference and phase difference between slots, the multiplying factor in case of contrast solution is lower than neat saline. For instance, in FIG. 13, (with contrast solution) antenna 111 comprises conductor length 112 of 1.15*λ_{eff} compared to the conductor length of 1.25*λ_{eff} of the antenna 71 (with neat saline) from FIG. 9.

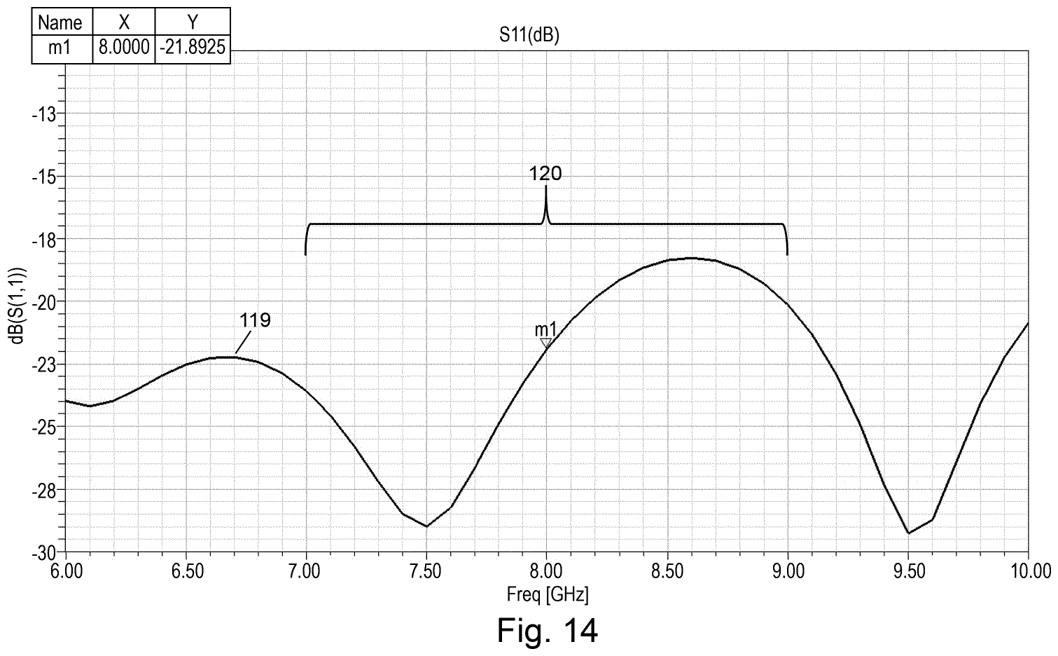

Alternatively, conductor lengths for each antenna can be evaluated using functions similar to equation 3. Slot widths are evaluated using the inverse attenuation fit method provided in this invention to achieve effective controlled and uniform radiation 117 in the vessel 25. S11 (dB) 119 performance of the antenna 111 is shown in FIG. 14 depicting high efficiency magnitude −21.89 dB wide bandwidth 120 and at 8 GHz.

The embodiments presented above may provide a robust system and method of designing and constructing a microwave antenna of any diameter including millimetre scale coaxial cables to provide uniform radiation distribution across a lesion of any length. In conditions such as different lesion length, fluid or any other variable that can influence antenna performance, the antenna can be easily optimised using the presented method to achieve uniform radiation.

Previously-known energy based and in particular microwave antennas designed to treat longer lesions may often create non-uniform zones with high and low energy depositions in the tissue [24, 25, 21]. High energy deposits may result in hot spots that may cause burns whereas low energy zones may leave untreated area of the lesions. The above embodiments may provide a method for depositing energy uniformly along the whole length of the lesion using microwave antenna design features such as non-uniform slot widths. Predictors such as lesion length, dielectric properties of the materials, effective wavelength (λ_{eff}) of the target material, frequency of operation, cable diameter etc. are used to derive the key design features of the antenna. Moreover, high and low energy zones in the treatment can be predicted thus providing a choice to the clinician to use an appropriate microwave antenna to achieve either uniform distribution or non-uniform distribution of energy depending upon type of condition, lesion and other factors.

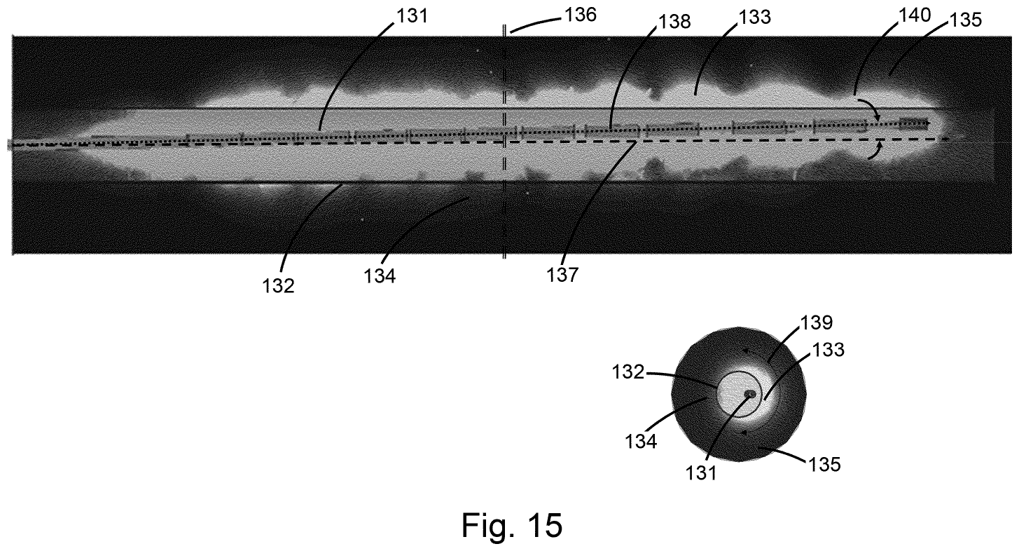

Other embodiments may provide a unilateral bias treatment feature. In FIG. 15, microwave antenna 131 is placed in the balloon 132 to provide hyperthermia to a tissue 135 (for example, muscle). The antenna 131 is deliberately placed at an angle 140 relative to the primary balloon axis 137, providing energy deposition 133 in only one of the side of the tissue 135 with low or no energy deposition 134 in the other side. Axes 137 (main axis) and 138 (angled axis) correspond to the balloon 132 and antenna 131 respectively. The angle a between axes 137 and 138 can vary depending upon the condition and area to be treated. Angle a can be any acute angle with a maximum limit value for example, sin⁻¹(R/L) where R is the radius of the balloon and L is the length of the antenna inside the balloon. For example, the angle a may be less than 10°, less than 5°, or less than 2°.

In other embodiments, the antenna may be placed parallel to the main axis of the balloon 132 and off centre to the balloon 132 and tissue. However, this may be difficult to manufacture as the parallel offset antenna may require tight bend radii that may damage the cable. The potential difficulty of manufacture may be addressed by placing the antenna at an angle such as shown in the FIG. 15.

The cross-sectional view along section line 136 shows a SAR biased area of a sector of approximately 180 degrees shown along arc 139 but could be divided into any ratio.

This feature may be beneficial to compensate for the change in radial proximity to the target tissue along the balloon axis by deliberately attenuating energy released. Another application of this feature can be to treat lesions preferentially keeping the healthy regions of the tissue untreated thus preserving natural anatomy of the tissue.

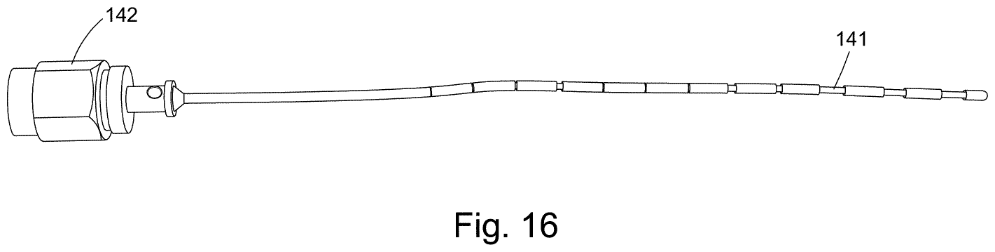

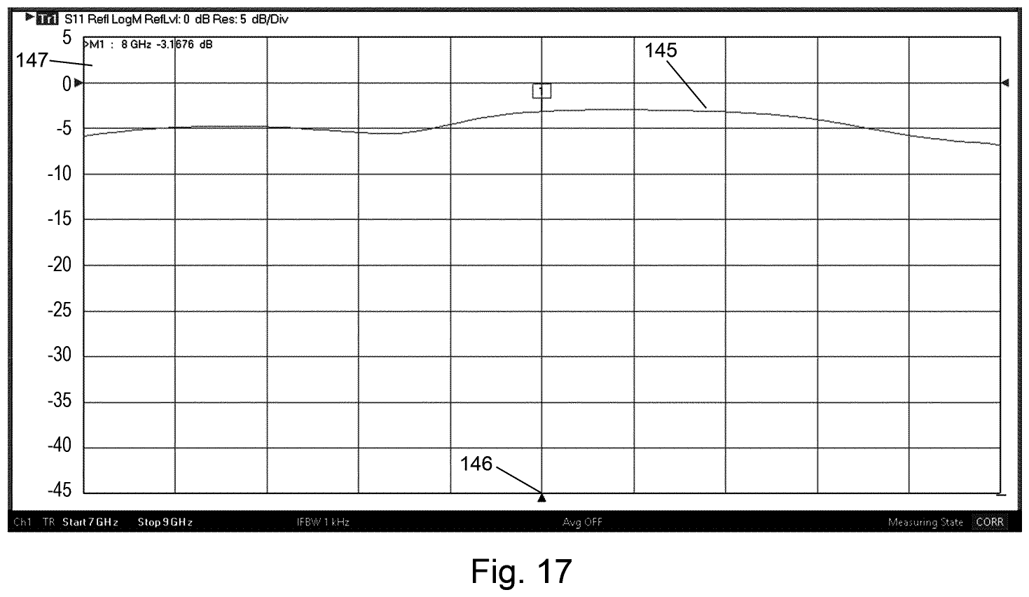

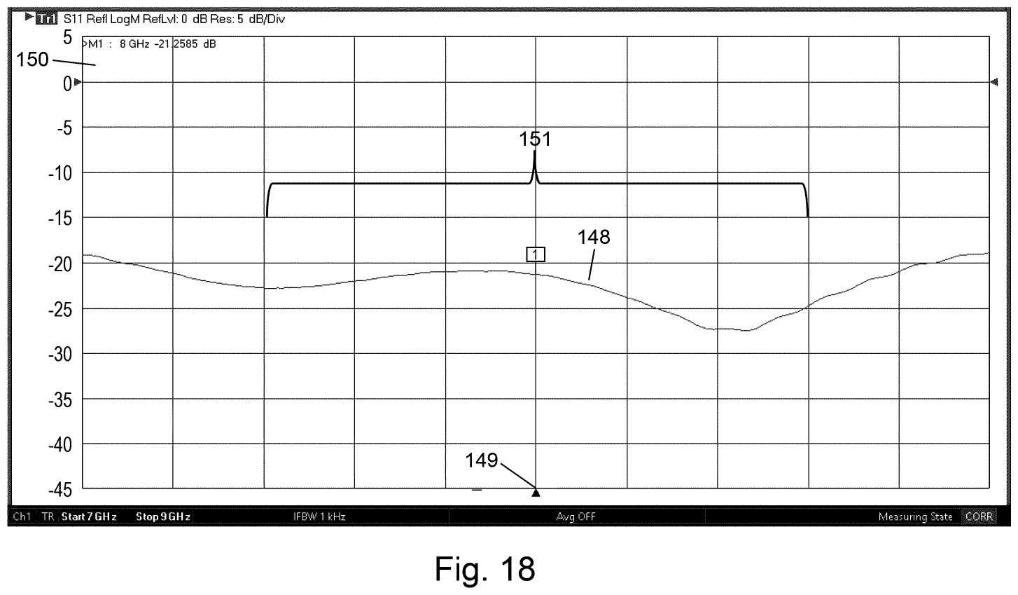

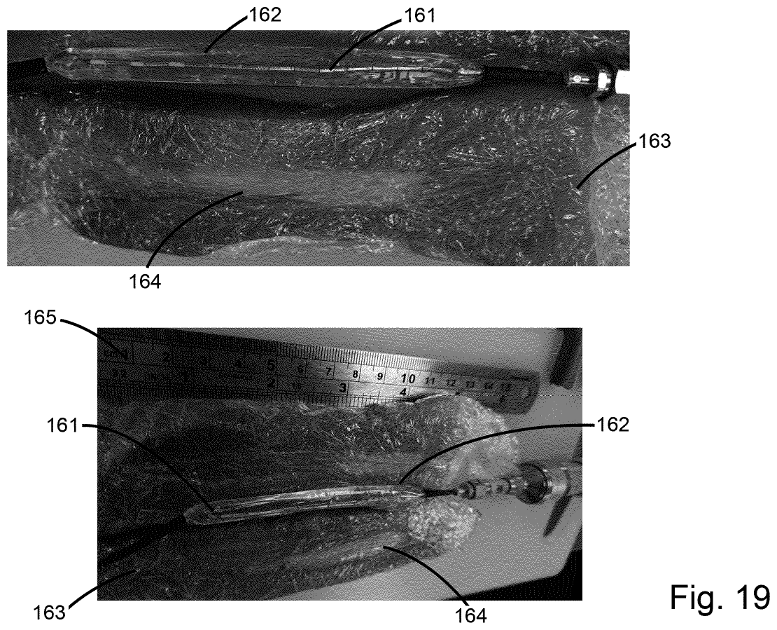

The microwave antenna presented above may be manufactured using a wide range of manufacturing methods such as but not limited to manual stripping, laser stripping, etching, mechanically and/or electrically driven cutting, high frequency blades, abrasive methods, thermal methods, chemical methods, pre-programmed stripping etc. In FIG. 16, an antenna is manufactured using coaxial cable Sucoform_43 with an outer diameter of 1.1 mm (Huber+Suhner, Herisau, Switzerland) are illustrated. The antenna is terminated using SMA connector 142 and comprises twelve annular slots 141 produced using a manual cut and peel method. Performance of the antennas in terms of S11 (dB) was tested using a VNA (Anritsu, Atsugi, Japan). Performance of the antenna of FIG. 16 is summarised in FIG. 17, FIG. 18 and FIG. 19. FIG. 17 shows antenna performance using S11 (dB) plot 145 in air. The magnitude of S11 (dB) is low (−3.17 dB) indicating poor radiation of the antenna into air. The poor S11 in air may provide safety in the case of accidental operation in air. A marker is placed on the 8 GHz datum 146 and the magnitude at 8 GHz is shown 147. FIG. 18 illustrates antenna performance S11 (dB) 148 when placed in 12% saline solution showing a match −21.26 dB 150 and substantially wide bandwidth 151.

Ex-vivo tests performed using the antenna in FIG. 16 are shown in FIG. 19. The antenna 161 was connected to a microwave generator operating at 8 GHz in accordance with the embodiments and was placed in a polyethylene balloon with 1:1 contrast and saline solution. The assembly was placed in porcine muscle tissue and microwave power of 20W was delivered for 1 minute (1200J). A uniform energy distribution 164 of approximately 70 mm was achieved. The measurement can be compared to the scale 165.

Antennas described above are designed for performing hyperthermia in biological tissues. A key application comprises providing hyperthermia to coronary, peripheral and all other blood vessels in the body. The antenna may be primarily applied in treating vascular conditions such as but not limited to atherosclerosis, carotid artery disease/stenosis/restenosis, chronic venous insufficiency (CVI), Varicose Veins, Deep Vein Thrombosis (DVT), calcification, Raynaud's Phenomenon, Renal Vascular Disease, abdominal aortic aneurysm (AAA), Thoracic Aortic Aneurysm, Buerger's Disease. Hyperthermia provided by antennas as described above may also be used as an adjuvant therapy to chemotherapy and radiation therapy in treating pre-cancerous or cancerous tumours and for ablating tumours that are surrounded by blood vessels. Further, antennas according to embodiments described above may be applied in treating any percutaneous angiographic, endovascular, endoscopic, keyhole, orthopaedic, stereotactic, gynaecologic, neurologic, neurovascular, glandular, urologic, pancreatic, abdominal condition or in any general therapeutic procedure for providing uniform and controlled microwave energy distribution over any length.

Antennas described above may be introduced into vessels in a minimally invasive manner. Antennas described above may be used to provide plaque modification. Since substantially uniform radiation may be provided along a substantial length of antenna (for example, 50 mm or more), the use of overlapping regions may be reduced. Inflation time may be reduced. The small diameter and larger length of the antenna may make it particularly suitable for the treatment of peripheral vascular disease.

It may be understood that the present invention has been described above purely by way of example, and that modifications of detail can be made within the scope of the invention.

Each feature disclosed in the description and (where appropriate) the claims and drawings may be provided independently or in any appropriate combination.

### REFERENCES

[1] “OMNIPAQUE™ (iohexol) Injection 140 180 240 300 350, FDA: Reference ID: 4080358,” General Electric, 2017. [Online]. Available: https://www.accessdata.fda.gov/drugsatfda_docs/label/2017/018956s099Ibl.pdf. [Accessed 29 5 2019].

[2] “Contrast Media,” General Electric, July 2018. [Online]. Available:

https://www. gehealthcare.co.uk/sitecore/content/gehc/home/products/contrast-media?sc_lang=en. [Accessed 29 5 2019].

[3] O. Mogabgab, V. Patel, T. Michael and K. A, “Impact of Contrast Agent Viscosity on Coronary Balloon Deflation Times: Bench Testing Results,” J Interv Cardiol, vol. 27, no. 2, pp. 177-181, 2014.

[4] G. Leibundgut, C. Degen and R. F, “Transcutaneous Puncture of an Undeflatable Coronary Angioplasty Balloon Catheter,” Case Reports in Cardiology, vol. Article ID 6252809, p. 5, 2018.

[5] J. Krueger, P. Ewert, S. Yilmaz, D. Gelernter, B. Peters, K. Pietzner, A. Bornstedt, B. Schnackenburg, H. Abdul-Khaliq, E. Fleck, E. Nagel, B. F and T. Kuehne, “Magnetic Resonance Imaging—Guided Balloon Angioplasty of Coarctation of the Aorta,” Circulation, vol. 113, no. 8, pp. 1093-1100, 2006.

[6] J. Ryan, W. Linde-Zwirble, L. Engelhart, L. Cooper and D. Cohen, “Temporal changes in coronary revascularization procedures, outcomes, and costs in the bare-metal stent and drug-eluting stent eras: results from the us medicare program.,” Circulation, p. 119:952-9, 2009.

[7] G. Tepe, U. Beschorner, C. Ruether, 1. Fischer, P. Pfaffinger, E. Noory and T. Zeller, “T. Drug-eluting balloon therapy for femoropopliteal occlusive disease: predictors of outcome with a special emphasis on calcium.,” J Endovasc Ther, vol. 22, p. 727-733., 2012.

[8] K. Katsanos, S. Spiliopoulos, P. Kitrou, M. Krokidis and D. Karnabatidis, “Risk of Death Following Application of Paclitaxel-Coated Balloons and Stents in the Femoropopliteal Artery of the Leg: A Systematic Review and Meta-Analysis of Randomized Controlled Trials.,” vol. 7, no. 24, p. e011245, 2018 .

[9] V. Ng, C. Mena, C. Pietras and L. AJ, “Local delivery of paclitaxel in the treatment of peripheral arterial disease.,” Eur J Clin Invest. , vol. 45, no. 5, pp. 333-45, 2015.

[10] D. Yin, M. Matsumura, j. Rundback, J. Yoho, B. Witzenbichler, G. Stone, G. Mintz and A. Maehara, “Comparison of plaque morphology between peripheral and coronary artery disease (from the CLARITY and ADAPT-DES IVUS substudies).,” Coron Artery Dis., vol. 28, no. 5, pp. 369-375, 2017.

[11] Gurm, C. Kasapis and S. Hitinder, “Current Approach to the Diagnosis and Treatment of Femoral-Popliteal Arterial Disease. A Systematic Review,” Curr Cardiol Rev., vol. 5, no. 4, p. 296-311., 2009.

[12] A. E. Simone Biscaglia, D. Bernucci, G. Bugani, E. Favaretto and G. Campo, “BRS implantation in long lesions requiring device overlapping: myth or reality?,” Journal of Thoracic Disease (Bioresorbable Vascular Scaffolds for the Treatment of Coronary Artery Disease)/, vol. 9 supp 9, 2017.

[13] U. Bildirici, M. Aktas, E. Dervis and U. Çelikyurt, “Mid-Term Outcomes of Stent Overlap in Long Total Occluded Lesions of Superficial Femoral Artery,” Med Sci Monit, vol. 23, pp. 3130-3135, 2017;.

[14] B. Alyavi and J. Uzokov, “Peripheral artery disease in the lower extremities: indications for treatment,” E-Journal-of-Cardiology-Practice, vol. 16, 2018.

[15] P. R. Kornaat, R. Sharma, R. J. v. d. Geest, H. J. Lamb, M. Kloppenburg, M.-P. H. I. Graverand, J. L. Bloem and I. Watt, “Positive association between increased popliteal artery vessel wall thickness and generalized osteoarthritis: is OA also part of the metabolic syndrome?,” Skeletal Radiology, vol. 38, pp. 1147-58, 2009.

[16] Y. G. Wolf, Z. Kobzantsev and L. Zelmanovich, “Size of normal and aneurysmal popliteal arteries: A duplex ultrasound study,” JOURNAL OF VASCULAR SURGERY, vol. 43, no. 3, pp. 488-492, 2006.

[17] R. Debasso, H. Astrand, N. Bjarnegård, A. R. Ahlgren and T. Sandgren, “The popliteal artery, an unusual muscular artery with wall properties similar to the aorta: Implications for susceptibility to aneurysm formation?,” JOURNAL OF VASCULAR SURGERY, vol. 39, no. 4, pp. 836-842, 2003.

[18] A. V. Telang, M. L. and M. Natarajan, “A study of the internal diameter of popliteal artery, anterior and posterior tibial arteries in cadavers,” Indian Journal of Clinical Anatomy and Physiology, vol. 3, no. 3, pp. 287-290, 2016.

[19] J. J. Dodge, B. Brown, E. Bolson and H. Dodge, “Lumen diameter of normal human coronary arteries. Influence of age, sex, anatomic variation, and left ventricular hypertrophy or dilation.,” Circulation, vol. 86, no. 1, pp. 232-46., 1992.

[20] ““Leaky” Cables Make Fine Broadband Antennas, microwavejournal,” 5 3 2015. [Online]. Available: https://www.gore.com/sites/g/files/ypyipe116/files/2016-05/Leaky%20Cables%20Make%20Fine%20Broadband%20Antennas%20_%202015-03-05%20_%20Microwave%20Journal.pdf. [Accessed 1 5 2019].

[21] S. MAINI and A. MARWAHA, “Development and Analysis of Coaxial Slot Antenna in Comparision with Coaxial Dipole Antenna for Interstitial Microwave Ablation,” in WORLD-EDU'12/CIT′WORLD-EDU'12/CIT'12 Proceedings of the 6th international conference on Communications and Information Technology, Athens, Greece, 2012.

[22] P. Gas, “Optimization of multi-slot coaxial antennas for microwave thermotherapy based on the S11-parameter analysis,” Biocybernetics and Biomedical Engineering, vol. 37, no. 1, pp. 78-93, 2017.

[23] Y. Jiang, J. Zhao, W. Li, Y. Yang, J. Liu and Z. Qian, “A coaxial slot antenna with frequency of 433 MHz for microwave ablation therapies: design, simulation, and experimental research,” Med Biol Eng Comput, vol. 55, no. 11, p. 2027-2036., 2017.

[24] M. F. J. C. Rubio, A. V. Hernandez, L. L. Salas, E. Avila-Navarro and E. Navarro, “Coaxial Slot Antenna Design for Microwave Hyperthermia using Finite-Difference Time-Domain and Finite Element Method,” The Open Nanomedicine Journal, vol. 3, pp. 2-9, 2011.

[25] C. Heng-mao, S. Ananda Mohan, T. Phillip, R. Leslie and G. Ramsay, “A Microwave Antenna For Medical Ablation”. European Patent Office Patent EP 1613230 B1, 2010.

[26] F. Suzuki, K. Takano, N. Ishii, J. Kumada, Y. Ozaki, Y. Nasu and K. Satou, “Leaky Coaxial Cable”. U.S. Pat. No. 8,378,764 B2, 2013.

[27] F. Rossetto, J. Brannan, J. Paulus and C. Debroski, “Leaky-wave Antennas For Medical Applications”. U.S. Pat. No. 8,202,270 B2, 2012.

[28] M. Sawarbandhe, B. Naik, V. Satpute and S. S, “Coaxial Antenna for Microwave Ablation,” in IEEE Distributed Computing, VLSI, Electrical Circuits and Robotics (DISCOVER), Mangalore, India, 2016.

[29] G. Karczewski, M. Kopcewicz and A. Kotlicki, “Skin depth effect in the RF collapse studies,” J. Phys. Colloques, vol. 41, pp. 217-218, 1980.

1. A microwave system, comprising:
a microwave generator;
a controller configured to control the microwave generator to generate microwave energy having a selected operational frequency or range of frequencies;
a microwave cable configured to deliver the microwave energy to a microwave antenna extending from or coupled to a distal end of the microwave cable; and
the microwave antenna comprising a plurality of slots distributed along a portion of the microwave antenna, wherein a width of the slots varies with distance along the portion of the microwave antenna.

2. The system according to claim 1, wherein the widths of the slots are selected such that, when microwave energy having a selected operational frequency or range of frequencies is delivered to the microwave antenna, a desired radiation profile is emitted by the microwave antenna.

3. The system according to claim 2, wherein the desired radiation profile comprises substantially uniform radiation along the portion of the microwave antenna.

4. The system according to claim 1, wherein at least one of (i), (ii), (iii) or (iv):
(i) the width of the slots increases with distance from the generator;
(ii) the slots are annular slots;
(iii) the cable is a coaxial cable and the slots are formed by removal of respective sections of an outer conductor of the coaxial cable; or
(iv) the slots are separated by conducting elements and a length of the conducting elements is selected to obtain a desired radiation profile.

5. (canceled)

6. (canceled)

7. (canceled)

8. (canceled)

9. (canceled)

10. The system according to claim 1, further comprising a balloon into which the microwave antenna is insertable.

11. (canceled)

12. (canceled)

13. The system according to claim 1, wherein at least one of (i) or (ii):
(i) the microwave antenna is configured to perform tissue hyperthermia at the operational frequency or range of frequencies; or
(ii) the microwave antenna is configured to perform plaque modification at the operational frequency or range of frequencies.

14. (canceled)

15. The system according to claim 1, wherein at least one of (i), (ii) or (iii):
(i) a diameter of the microwave antenna and a diameter of the cable are selected such as to be insertable into a vessel to be treated;
(ii) a diameter of the microwave antenna is less than 10 mm, or
(iii) the portion of the microwave antenna comprises at least 50 mm of the microwave antenna length.

16. (canceled)

17. (canceled)

18. (canceled)

19. A microwave antenna comprising a plurality of slots distributed along a portion of the microwave antenna, wherein a width of the slots varies with distance along the portion of the microwave antenna, wherein the widths of the slots are selected such that, when microwave energy having a selected operational frequency or range of frequencies is delivered to the microwave antenna, a desired radiation profile is emitted by the microwave antenna.

20. A method of performing a tissue heating process comprising:
positioning a microwave antenna in or adjacent to a treatment area, the microwave antenna comprising a plurality of slots distributed along a portion of the microwave antenna, wherein a width of the slots varies with distance along the portion of the microwave antenna; generating microwave energy by a microwave generator, the microwave energy having a selected operational frequency of range of frequencies;
providing the microwave energy to the microwave antenna; and
heating the treatment area by radiation of microwave energy from the microwave antenna.

21. The method according to claim 20, wherein positioning the microwave antenna in or adjacent to the treatment area comprises inserting the microwave antenna into a balloon and inserting the balloon into a vessel.

22. The method according to claim 21, wherein at least one of (i), (ii), (iii) or (iv):
(i) the microwave antenna is inserted into the balloon so that a longitudinal axis of the microwave antenna is angled relative to a longitudinal axis of the balloon;
(ii) the microwave antenna is configured to be inserted into the balloon so that a longitudinal axis of the microwave antenna is offset from a longitudinal axis of the balloon;
(iii) the vessel is a peripheral vessel; or
(iv) the widths of the slots are selected such that, when microwave energy having a selected operational frequency or range of frequencies is delivered to the microwave antenna, a desired radiation profile is emitted by the microwave antenna into the treatment area.

23. (canceled)

24. (canceled)

25. (canceled)

26. (canceled)

27. The method according to claim 20, wherein at least one of (i), (ii) or (iii):
the treatment area comprises a lesion;
(ii) the tissue heating is such as to cause tissue hyperthermia in the treatment area; or
(iii) the tissue heating is such as to cause plaque modification in the treatment area.

28. (canceled)

29. The method according to claim 20, wherein the balloon contains a fluid and at least one of (i), (ii) or (iii):
(i) the fluid has at least one of a desired value of dielectric constant, a desired loss tangent or electrical conductivity;
(ii) the fluid comprises at least one of deionised water, reverse osmosis water or saline, optionally a combination of contrast agent and one or more of deionised water, reverse osmosis water or saline; or
the heating is performed for a treatment time, optionally a treatment time longer than one minute.

30. A method for designing a microwave antenna, the method comprising:
a) simulating radiation from an initial antenna design, the initial antenna design comprising a plurality of slots arranged along a portion of a coaxial cable;
b) fitting the simulated radiation to an attenuation curve;
c) using an inverse of the attenuation curve to determine a respective slot width for each of the plurality of slots; and
d) designing a microwave antenna having the determined slot widths.

31. A method according to claim 30, wherein the slot widths are such as to provide a desired radiation profile when microwave energy is supplied to the microwave antenna.

32. (canceled)

33. The method according to claim 30 wherein at least one of (i) or (ii):
the plurality of slots of the initial antenna design are equally sized along the coaxial cable; or
(ii) the plurality of slots of the initial antenna design are equally spaced along the coaxial cable.

34. The method according to claim 30, wherein at least one of (i), (ii) or (iii):
(i) the simulating of the radiation comprises simulating radiation into a predetermined material having a known relative permittivity;
(ii) the plurality of slots are separated by conducting elements, each conducting element being of the same size, and the method further comprises determining a size of the conducting elements based on the relative permittivity and a length of the microwave antenna; or
(iii) using an inverse of the attenuation curve to determine a respective slot width for each of the plurality of slots comprises determining a leakage factor function from the attenuation curve and using the leakage factor function to determine the slot widths.

35. (canceled)

36. (canceled)

37. (canceled)

38. The method according to claim 30, further comprising iteratively repeating steps a) to c) until a desired radiation pattern is achieved.

39. (canceled)

40. (canceled)

41. A method of fabricating a microwave antenna, the method comprising:
providing a coaxial cable; and
at a distal end of the coaxial cable, selectively removing a plurality of sections of an outer conductor of the coaxial cable to expose sections of the inner conductor, thereby forming a plurality of radiating slots, wherein slot widths of the radiating slots are determined using a method in accordance with claim 30.

42. The method according to claim 41, wherein the sections removed from the outer conductor are annular sections, and the slots are annular slots.
